(19) United States
(12) Patent Application Publication
(10) Pub. No.: US 2017/0082640 A1
(43) Pub. Date: Mar. 23, 2017

(54) Label-Free Single and Multi-Photon Fluorescence Spectroscopy to Detect Brain Disorders and Diseases: Alzheimer, Parkinson, and Autism From Brain Tissue, Cells, Spinal Fluid, and Body Fluids

(71) Applicant: Robert R. Alfano, Bronx, NY (US)

(72) Inventors: ROBERT R. ALFANO, Bronx, NY (US); Lingyan Shi, New York, NY (US)

(21) Appl. No.: 15/272,086

(22) Filed: Sep. 21, 2016

Related U.S. Application Data
(60) Provisional application No. 62/284,132, filed on Sep. 21, 2015.

(51) Int. Cl. G01N 33/68; G01N 21/64; A61B 10/00; A61B 5/00; A61B 10/02

(52) U.S. Cl. CPC G01N 33/6896; A61B 5/4064; A61B 10/02; A61B 10/0045; A61B 5/4088; A61B 5/0071; A61B 5/4082; G01N 21/6486; G01N 21/645; A61B 2010/0077; G01N 2800/2821; G01N 2800/2835; G01N 2800/28; G01N 2021/6471; G01N 2201/0621

(57) ABSTRACT

A label free single or multi-photon optical spectroscopy for measuring the differences between the levels of fluorophores from tryptophan, collagen, reduced nicotinamide adenine dinucleotide (NADH). and flavins exist in brain samples from a of Alzheimer's disease (AD) and in normal (N) brain samples with label-free fluorescence spectroscopy. Relative quantities of these molecules are shown by the spectral profiles of the AD and N brain samples at excitation wavelengths 266 nm, 300 nm, and 400 nm. The emission spectral profile levels of tryptophan and flavin were much higher in AD samples, while collagen emission levels were slightly lower and NADH levels were much lower in AD samples. These results yield a new optical method for detection of biochemical differences in animals and humans for Alzheimer's disease.

### BACKGROUND OF THE INVENTION

1. Field of the Invention

The invention generally relates to diagnostic testing of brain disorders and diseases and, more specifically to label-free one or multiple photon-emission (“PE”) such as IPE, 2PE and 3PE fluorescence (“PEF”) spectroscopy to detect brain disorders and diseases: Alzheimer, Parkinson and autism from brain tissue, cells, spinal fluid, and body fluids.

2. Description of Prior Art

Alzheimer's disease (AD), a degenerative disorder that attacks neurons in the brain and leads to the loss of proper cognition, ravages the lives of millions of people all across the world. It is the sixth leading cause of death in the United States. Although the disease has been the focus of much scientific research in past years, there still is no cure; and from 2000-2010 the proportion of deaths resulting from Alzheimer's disease in America has gone up 68%. [1] A large proportion of people with Alzheimer's disease remained undiagnosed. However, early diagnosis can help them make decisions for the future while it is still possible to do so, and can allow people to receive early treatment to improve their cognition and increase the quality of their life as they live with Alzheimer's disease. [2]

Physicians diagnose Alzheimer's disease with just an examination of a patient's state, inquiries into the familial history of psychiatric and neurological disorders, and a neurological exam.[1] Other newer methods of diagnosis include Magnetic Resonance Imaging (MRI) to look for Hippocampal atrophy,[3] Positron Emission Tomography (PET) scans, [4] and examining levels of beta-amyloid and tau protein in cerebrospinal fluids taken from the patient.[5]

Scientists continue to search for a better method to detect AD. Label-free optical spectroscopy offers a new tool to detect and understand the AD brain at the molecular level. In 1984, Robert R. Alfano and his group of researchers at the City College of New York (C.C.N.Y.) pioneered the use of optical spectroscopy to detect cancer by looking at the native fluorescence levels of organic biomolecules.[6] This process of biomedical imaging, using light and the native 1PE, 2PE and 3PE fluorescence of certain proteins and molecules within human tissue, has been expanded upon and applied to examine levels of tryptophan, NADH, flavin, and collagen in normal and cancerous breast tissue for diagnosing certain types of cancer.[7,8]

Tryptophan, NADH, collagen, and some other molecules have been examined as potential markers of Alzheimer's disease; Optical spectroscopy has not been employed to study the linear fluorescence of these biomarkers excited at various wavelengths in AD and normal (N) brain tissue The focus of this study is to apply optical fluorescence spectroscopy for measuring fluorescence levels of key biomolecules (tryptophan. NADH, collagen, and flavin) in AD and N brain tissues using a mouse model of AD, and to propose a potential method for detection and diagnosis of Alzheimer's disease in humans. Different amounts of these label free biomolecules in Brain are shown in FIGS. 1(a)-1(c) for different excitation wavelengths from 266 to 400nm. These fluorescence spectral difference forms the teachings for the claims.

“Optical Biopsy” is a novel method using Raman and fluorescence spectroscopy at selected wavelengths to diagnose disease such as cancer, atherosclerosis, and brain disease without removing tissue from body, offering a new armamentarium. Key native molecules in tissues reveal the differences between diseased and normal tissues of various organs due to morphological and molecular changes in the tissue. The key label free optical methods are: fluorescence and Raman spectroscopies. Various human tissue types (prostate, breast, lung, colon, arteries, and gastrointestinal) have been studied using optical biopsy. One can use lamps or LEDs to excite 1 PEF and femtosecond laser (Ti) for 2 PEE and 3 PEF processes.

### SUMMARY OF THE INVENTION

We teach here the use of Linear and Nonlinear Optical Biopsy Spectroscopy to study brain and its disorders such as Alzheimer, Parkinson and Autism among others.

Optical spectroscopy has been considered a promising technique for cancer detection for more than two decades because of its advantages over the conventional diagnostic methods: no tissue removal, minimal invasiveness, less time consumption and reproducibility. Optical Biopsy was first used by Alfano et al., in 1984, who measured label free native fluorescence (NF), also called autofluorescence. Human tissue is mainly composed of an extracellular matrix of collagen fiber, proteins, fat, water, epithelial cells, which contains a number of key fingerprint native endogenous fluorophore molecules: tryptophan, collagen, elastin, reduced nicotinamide adenine dinucleotide (NADH), flavin adenine dinucleotide (FAD) and porphyrins. Tryptophan is an amino acid required by all forms of life for protein synthesis and other important metabolic functions, accounting for the majority of protein fluorescence. NADH and FAD are involved in the oxidation of fuel molecules and can be used to probe changes in cellular metabolism. It is well known that abnormalities in metabolic activity precede the onset of many diseases: carcinoma, diabetes, atherosclerosis, brain and Alzheimer's disease. The photonic tools use fiber spectroscopic ratiometer, fiber-optic endoscope for in vivo use for detecting in situ brain disorders pumped by linear and multiphoton excitation.

### BRIEF DESCRIPTION OF THE DRAWINGS

The above and other aspects, features and advantages of the present invention will be more apparent from the following description when taken in conjunction with the accompanying drawings, in which:

FIGS. 1(a)-1(c) show spectral profiles of AD and N brains at excitation wavelength (a) 266 nm, (b) 300 nm, and (c) 400 nm, respectively;

FIGS. 2(a) and 2(b) show absorption and fluorescence profiles of key biomolecules, respectively, FIG. 2(a) showing absorption of key molecules, and FIG. 2(b) showing emission of key molecules;

FIGS. 3(a) and 3(b) show fluorescence spectroscopy electronic states and elastic and Raman “vibrational states” for two-photon deeper tissue imaging;

FIGS. 4(a)-4(d) show native SHG, 2PEF, 3PEF and 4PEF Label Free (native molecules) emissions; and

FIG. 5 illustrates ICG absorption in relation to Soret peaks or bands.

### DETAILED DESCRIPTION

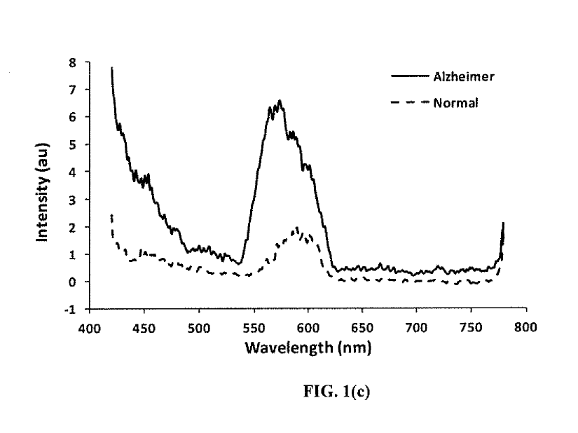

Fluorescence spectroscopy measures allowed electronic transitions of various chromophores in the complex tissue structure. There are several natural label free fluorophores that exist in tissue and cells which, when excited with ultraviolet light, emit fluorescence in the ultraviolet and visible regions of the spectrum. Some of the absorption and emission spectra of these native endogenous fluorophore molecules are shown in FIGS. 2(a)-(b). The Flavins and NADH show changes in the spectra between their oxidized and reduced state. The relatively large emission intensity from tissues and the need of broadly tunable excitation sources in the UV and visible has led researchers to develop lamp based fluorescence systems instead of lasers and now LEDs from 260 nm to 550 nm to excite the key biomolecules for I PER These states can be excited by 1 PEF which is more a surface process and 2 PEF or 3 PEF for deeper penetration.

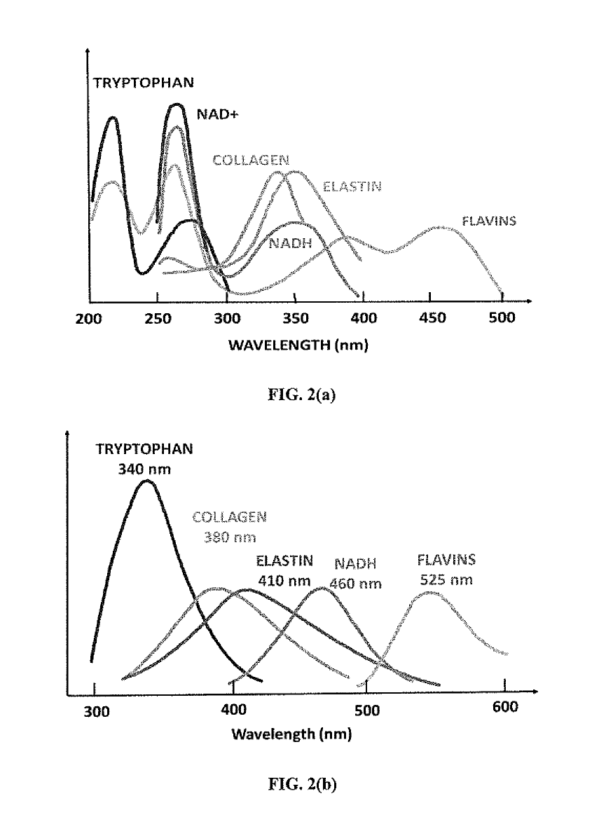

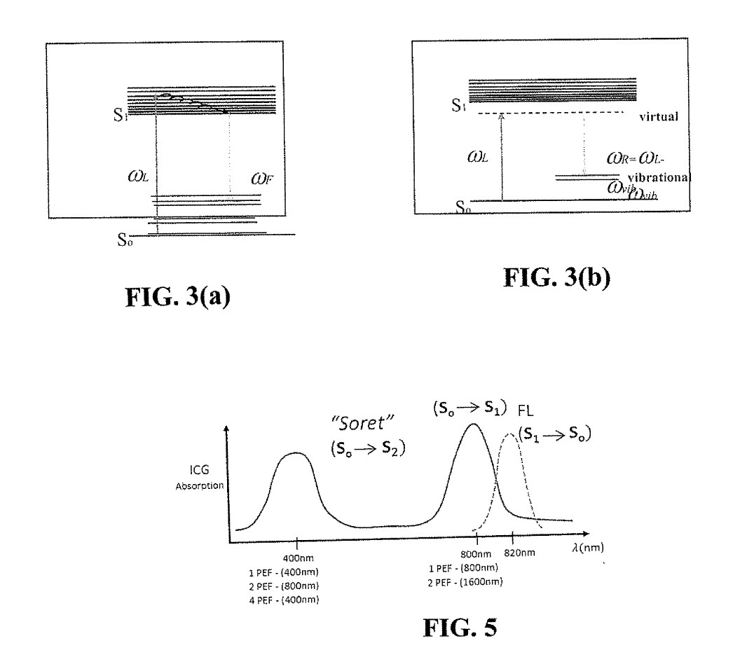

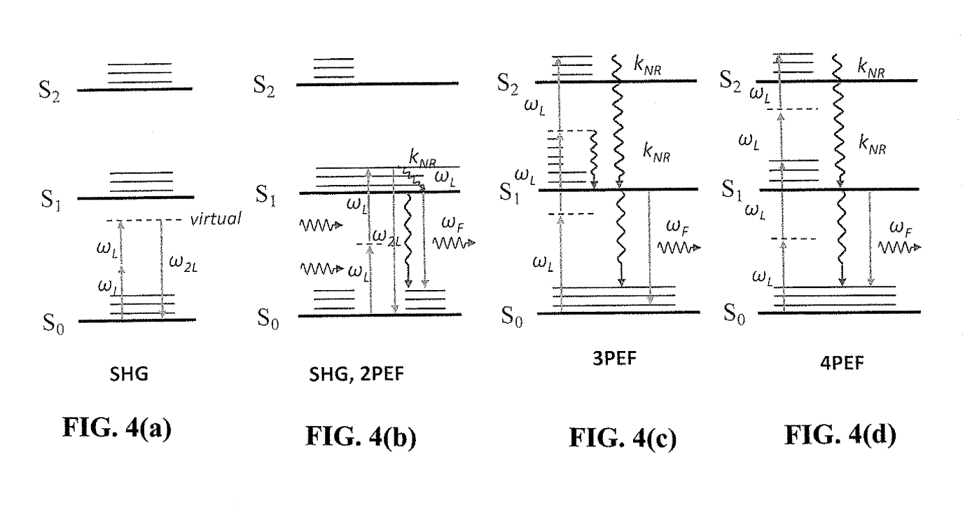

A basic fiber unit incorporates a fluorescence section and uses LEDs at 260 nm, 280 nm 300 nm, 350 nm, and 400 nm to excite Tryptophan, collagen, elastin, NADH, and FAD in brain disease. Femtosecond Ti lasers (700 nm to 1200 nm) can be used to excite the Key molecules (3 PEF for tryptophan a 267 nm); and 2 PEF for collagen, NADH and flavins. See FIGS. 3(a)-5.

Significant differences of emission peaks were found in these molecules in AD and normal (N) brain. The fluorescence intensity levels from tryptophan: AD>N; from collagen: AD˜N; from NADH: N>AD and from flavin: AD>N. These observation provides effective techniques to explore an optical diagnosis of Alzheimer's disease by examining the spectral profiles of various molecules in brain tissue, eye fluid, body fluids, and /or spinal fluid ex vivo and in vivo using optical fibers.

### Materials and Methods for Proof of Concept

### Animal Preparation

Mice were purchased from Jackson Laboratory and housed at the City College Animal Facility. A 2-month-old triple transgenic AD mouse harboring PS1M146V, APPSwe and tauP301L transgenes in a uniform strain background was used. Another N mouse at the same age was used as control.

The mouse was anesthetized with a mixture of ketamine and xylazine (41.7/2.5 mg/kg body weight), then was decapitated and the brain was dissected and post-fixed overnight with 4% formaldehyde in 0.1 M phosphate buffer (PB) and subsequently immersed in 30% sucrose in 0.1 M PB for up to 48 hrs prior to slicing. The hippocampus of both AD and N brains was sliced coronally at a thickness of 1 mm, by using a brain matrix (RWD Life Science Inc, San Diego, Calif.), and was placed in a cuvette (Sigma-Aldrich, St. Louis, Mo.).

### Basic Theory of Fluorescence

It is well known that the fluorescence intensity I_{f} depends on efficiency Q from the radiative rate Kr and nonradiative rate Knr, the relationship can be written as:

Q=Kr/(Kr+Knr) (1)

Eq (1) for Q equals to the ratio of numbers of photons emitted out to the numbers of photon pumped in (Nout/Nin). The intensity from excited molecules I_{f} is

I_{f}=Ω/4π(Q·N), (2)

where Ω is the solid angle and N is the number of excited molecules. Q value. The Knr depends on the interaction of molecules with their host environments. Weak interaction will lead to a small Knr and give more emission intensity. When Knr>>Kr the emission is reduced.

### LS 50 Fluorescence Spectrometer

The fluorescence of Alzheimer and N brain tissues was measured by a LS 50 fluorescence spectrometer (PerkinElmer, Waltham, Mass.). A xenon lamp was used as the discharge light source in the spectrometer. There are two monochromators, with the excitation monochromator able to detect light ranging from 200-800 nm and the emission monochromator able to detect light ranging from 200-650 nm. Pulsed light from the xenon lamp hits a diffraction grating, which selects the wavelength being used. This light then enters through the excitation monochromator, at which point the light strikes the sample, which is stored in a cuvette and positioned between the two monochromators. After being struck by the light at the selected wavelength, the sample fluoresces, and the fluorescence light is collected on the other side through the emission monochromator. The wavelength accuracy is +/−1 nm and the slit widths can be varied 2.5 nm-15 nm and 2.5-20 nm for the excitation and emission slit, respectively.

The AD and N brain samples were excited at wavelengths 266 nm, 300 nm, and 400 nm, to examine the fluorescence peaks of each of tryptophan, NADH, FAD, and collagen. All measurements were performed by using a scanner (at 100 nm/sec), and the samples were held in cuvettes during the measurement.

A 300 nm or 400 nm filter was placed in between the excitation monochromator and the sample for scans at 300 nm or 400 nm respectively, whereas the scan at 266 nm was done without a filter. The measurements of the AD and N brain samples were each taken twice with different slit widths at each excitation wavelength. The slit widths for the scans at 300 nm and 400 nm were 7 nm and 5 nm respectively for the first round of measurements, and 5 nm and 4 nm respectively for the second round. Due to a lack of the filter at 266 nm, the excitation and emission slit widths were 4 mm and 3 mm respectively for the first round of measurements, and 3 mm excitation and 2.5 mm emission for the second round.

### Results and Discussion

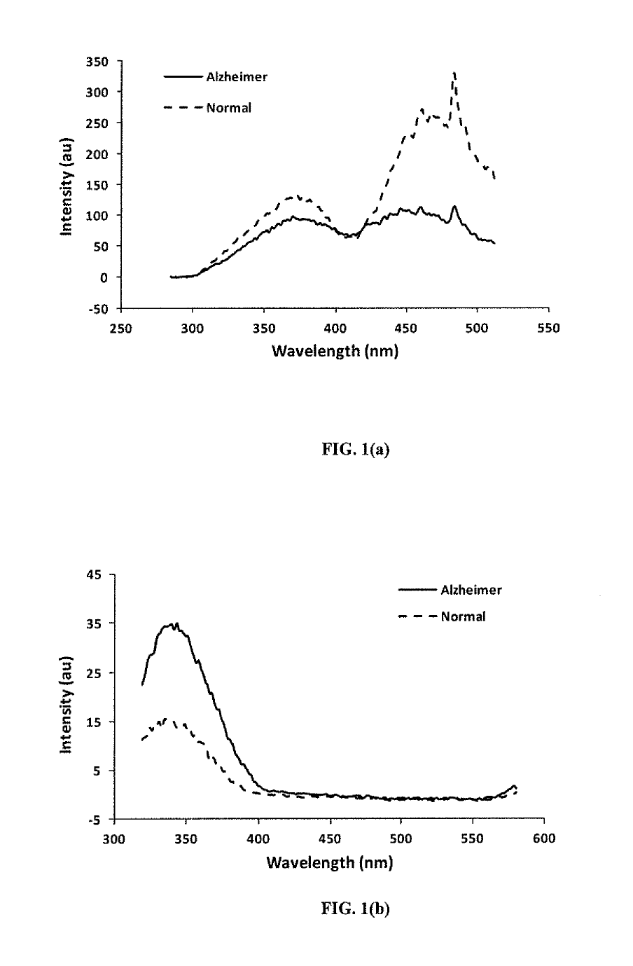

The present study is aimed at detecting AD by measuring fluorescence intensities of multiple biomolecules, we used N and AD brain samples from mice. FIG. 1 displays the fluorescence spectral profiles in AD and N brain samples at the excitation wavelengths 266 nm (FIG. 1a), 300 nm (FIG. 1b), and 400 nm (FIG. 1c). Different excitation wavelengths were employed to determine the emission spectra of each biomolecule (tryptophan, collagen, NADH, and flavin), as shown in FIGS. 2(a) and 2(b). Table 1 summarizes the emission wavelengths for assigned molecules at peak emissions in AD and N brain tissues under different excitation wavelengths. One can use 1 PEF, 2 PEF and 3 PEF to excite the molecules in Table 1,

TABLE 1
Emission peaks in Alzheimer and N brain samples

| Excitation | | Peak | Peak | Substance |
|---|---|---|---|---|
| wavelength | Tissue* | wavelength | intensity | Excited |
| 266 nm | AD | 370 | 96.92 | Collagen |
| | | 460 | 111.5 | NADH |
| | N | 372.5 | 132.1 | Collagen |
| | | 461.5 | 270.4 | NADH |
| 300 nm | AD | 334 | 34.87 | Tryptophan |
| | N | 335.5 | 15.58 | Tryptophan |
| 400 nm | AD | 453.5 | 3.9 | NADH |
| | | 573.5 | 6.55 | flavin |
| | N | 447.5 | 1.17 | NADH |
| | | 573.5 | 1.97 | flavin |

*AD: Alzheimer; N: normal.

FIG. 1(a) shows that at excitation 266 nm the fluorescence peaks of AD and N brain tissues are at the same wavelengths (ranging 365-385 nm and 460-490 nm), corresponding to the wavelengths of emission peaks of collagen and NADH respectively;

Peak intensities in AD brain are 73% (collagen) and 41% (NADH) respectively of those in N brain (Table 1). The levels of collagen in AD and N brains are relatively close, making it difficult to distinguish AD from N brain in this respect. An alternate way to differentiate the spectral profiles in AD or N brain is to compare the ratio of NADH intensity to collagen intensity, which is ˜1:1 in AD brain and 2:1 in N brain. Comparing the spectral profiles (peaks) of collagen and NADH and their relative ratio may be an applicable method for diagnosing Alzheimer's disease.

The scans at excitation wavelength 300 nm offer diagnostic possibilities for AD. The emission intensities of the AD and N brain tissues both peak in the range of 330-350 nm (FIG. 1b), which match the wavelength of the emission peak of tryptophan in FIG. 2(b). In addition, the peak. intensity of tryptophan in AD brain tissue is 2.2 times higher than that in N brain tissue (Table 1). Tryptophan, due to its properties of native fluorescence, has been employed in a vast array of biomedical imaging processes, including the diagnosis of breast cancer and other types of cancer. This vast disparity of tryptophan fluorescence levels in AD and N mouse brain scans proposes another method for AD diagnosis. It appears that tryptophan has more Kr or less Knr which may be due to the tissue environment.

The scan taken at excitation wavelength of 400 nm excited flavin in AD and N brains. In both AD and N brain tissues, the wavelength of peak emissions were found in the range of 560-580 in (FIG. 1c), consistent with the emission wavelength of NADH and flavin in FIG. 2(b). The peak intensity of NADH and flavin are both 3.3-fold higher in AD brain compared to N brain (Table 1).

It appears that tryptophan emission efficiency is more in AD than N which may be due to fewer interactions to the host molecules in the environment in AD brain tissue and the nonradiative Knr interaction was reduced or Kr was increased . The significant difference of flavin emission peaks, in addition to the fact that the excitation wavelength at 400 nm is less harmful to cells than shorter wavelength, makes scans at 400 nm another promising prospect for Alzheimer's diagnosis, especially in combination with the scans at excitation wavelengths 266 nm and 300 nm as discussed above. The future direction could use time resolved fluorescence which gives fluorescence rate (K_{f}=Kr+Knr) and combines with longer wavelength multiphoton excitation which offers deeper tissue penetration.

This current study is the first teaching to investigate the fluorescence spectra of collagen, NADH, tryptophan, and flavin in Alzheimer and N brain tissues of a mouse model for human brain . It demonstrates significant differences of emission peaks of these molecules in AD and N brain. The fluorescence intensity levels from tryptophan: AD>N; from collagen: AD˜N; from NADH: N>AD and from flavin: AD>N. This work provides effective techniques to explore diagnosis of Alzheimer's disease by examining the spectral profiles of various biomolecules.

### REFERENCES

1. Alzheimer's Association, “2013 Alzheimer's disease facts and figures,” Alzheimer's and Dementia 9 (2). 208-245 (2013).
2. Alzheimer's Disease International, “World Alzheimer Report 2011: The benefits of early diagnosis and intervention,” London (2011).
3. L. A. van de Pol, A. Hensel, W. M. van der Flier, P-J. Visser, Y. A. L. Pijnenburg, F. Barkhof, H. J. Gertz, and P. Scheltens, “Hippocampal atrophy on MRI in frontotemporal lobar degeneration and Alzheimer's disease,” J. Neurol. Neurosurg. Psychiatry 77 (4), 439-442 (2006).
4. G. E. Alexander, K. Chen, P. Pietrini, S. I. Rapoport, E. M. Reiman, “Longitudinal PET Evaluation of Cerebral Metabolic Decline in Dementia: A Potential Outcome Measure in Alzheimer's Disease Treatment Studies,” The American J. of Psychology 159 (5), 738-745 (2002).
5. F. Hulstaert, K. Blennow, A. Ivanoiu, H. C. Schoonderwaldt, M. Riemenschneider, P. P. De Deyn, C. Bancher, P. Cras, J. Wiltfang, P. D. Mehta, K. Iqbal H. Pottel, E. Vanmechelen, and H. Vanderstichele, “Improved discrimination of AD patients using beta-amyloid(1-42) and tau levels in CSF,” Neurology 52 (8), 1555-1562, (1999).
6. R. R. Alfano, D. Tata, J. Corder° , P. Tomashefsky, F. Longo, and M. Alfano, “Laser induced fluorescence spectroscopy from native cancerous and normal tissue,” IEEE J. Quantum Electron. 20, 1507-1511 (1984).
7. Y. Pu, W. Wang, Y. Yang, and R. R. Alfano, “Native fluorescence of human cancerous and normal breast tissues analyzed with non-negative constraint methods,” Applied Optics 52 (6), 1293-1301 (2013).

8. L. A. Sordillo, P. P. Sordillo, Y. Budansky, Y. Pu, and R. R. Alfano, “Differences in fluorescence profiles from breast cancer tissues due to changes in relative tryptophan content via energy transfer: tryptophan content correlates with histologic grade and tumor size but not with lymph node metastases,” J. of Biomedical Optics 19 (12), 125002-1-125002-6 (2014).

1. Method of detecting brain disorders and disease comprising the steps of
collecting a sample of cells and/or tissue from a group consisting of brain tissue, eye fluid, body fluid and/or spinal fluid containing molecules found in a brain being examined (AZ) and from a normal brain (N);
exposing and exciting said molecules to selected wavelengths within the range of 200-800 nm by 1 PEF and/or by 700 nm to 1200 nm ultrafast laser pulses (30 to 300 fs) by 2 PEF and 3 PEF;
detecting emission of fluorescence from the excited molecules;
examining fluorescence peaks of each of tryptophan, NADH, flavins and collagen;
comparing intensity levels of excitation and emission spectra for tryptophan, collagen, NADH and flavin; and
establishing a diagnosis of Alzheimer's disease when the fluorescence intensity levels from a brain being examined (AD) and a normal brain (N) satisfy at least the following relationships:

| | |
|---|---|
| Collagen | AD~N |
| NADH | N > AD. |

2. A method as defined in claim 1, wherein exposure wavelengths cover the range of 260 nm to 500 nm.

3. A method as defined in claim 1, wherein exposure wavelengths cover the range of 320 nm to 550 nm.

4. A method as defined in claim 1, wherein the following relationships are considered in establishing the presence or absence of brain disorder or disease:

| | |
|---|---|
| Tryptophan | AD > N |
| Collagen | AD~N |
| NADH | N > AD |
| Flavin | AD > N. |

5. An optical radiometer for detecting brain disorders and disease comprising:
a spectrometer optical analyzer at fixed wavelengths;
a source for exciting a sample of molecules in cells and/or tissue within the range of 200 nm 800 nm by 1 PEF and/or by 700 nm to 1200 nm ultrafast laser pulses (30 to 300 fs) by 2 PEF and 3 PEE; and
photo detectors for detecting fluorescence peaks of each of tryptophan, NADH, Flavins and collagen emitted from said molecules, said spectrometer optical analyzer including means for measuring the differences in the levels from native biomarkers of tryptophan, collagen, NADH and Flavin, whereby the presence of Alzheimer, Parkinson, and Autism can be established when at least the following relationships are found:

| | |
|---|---|
| Collagen | AD~N |
| NADH | N > AD. |

6. An optical radiometer as defined in claim 5, wherein optical fibers with photodetectors are used for detecting said optical peaks.

7. An optical radiometer as defined in claim 6, wherein said photodetectors are selected from a group comprising CMOS, PMT and CCD.

8. An optical radiometer as defined in claim 5, wherein spectral units are used to directly probe and excite different areas of the brain.

9. An optical radiometer as defined in claim 8, wherein said spectral units are selected from a group comprising spectrograph, spectrometer and optical filters.

5. optical radiometer as defined in claim 5, wherein said source of excitation is selected from a group comprising xenon lamps. LEDs and femtosecond lasers for nonlinear 2 PEF and 3 PEF.

11. An optical radiometer as defined in claim 10, further comprising a diffraction grating for intercepting the output of said source of excitation to provide desired excitation wavelengths for linear and non-linear 2 PEF and 3 PEF.

12. An optical radiometer as defined in claim 5, further comprising an excitation monochromator arranged between said source of excitation and the sample for detecting and transmitting light within the range of 200-800 nm.

13. An optical radiometer as defined in claim 5, further comprising and emission monochromator for detecting emissions from the sample within the range of 200-650 nm.

14. An optical radiometer as defined in claim 5, wherein the sample is maintained in a cuvette.

15. An optical radiometer as defined in claim 14, Wherein excitation and emission monochromators are provided with said cuvette being positioned between said excitation and emission monochromators.

16. An optical radiometer as defined in claim 5, wherein said source for exciting comprises UV LEDs for 280 nm to 500 nm 1 PEF.
